(19) United States
(12) Patent Application Publication
(10) Pub. No.: US 2006/0264700 A1
(43) Pub. Date: Nov. 23, 2006

(54) ADULT SEXUAL AID DEVICE

(76) Inventor: Buddy Jefferson Guy, Las Vegas, NV (US)

Correspondence Address: KENEHAN & LAMBERTSEN, LTD;JOHN C LAMBERT, 1771 E. FLAMINGO ROAD, SUITE 117B, LAS VEGAS, NV, 89119-0839, US

(21) Appl. No.: 11/419,982

(22) Filed: May 23, 2006

Related U.S. Application Data
(60) Provisional application No. 60/683,913, filed on May 23, 2005.

(51) Int. Cl. A61F 5/00

(52) U.S. Cl. 600/38

(57) ABSTRACT

A sexual aid utilizes a compressible rod to simulate the reciprocating motion associated with certain types of sexual activity. An adult toy is attached to an outwardly extending support arm, and a handle is attached to the rod. Through use of the handle a user can apply a downward force on the compressible rod in a rhythmic manner, causing the compressible rod to oscillate between a compressed and an extended configuration—as does the attached sex toy.

### CROSS-REFERENCE TO RELATED APPLICATION

This application claims the benefit of U.S. Provisional Patent Application, Ser. No. 60/683,913, filed May 23, 2005.

### TECHNICAL FIELD

The embodiments of the invention disclosed herein relate to adult sexual apparatus, including novelty apparatus for personal use, the apparatus permitting a user to perform various sexual exercises in stimulating and healthful manners.

### BACKGROUND ART

Therapeutic aids and mechanisms utilized to improve sexual relations between sexual partners are known in the art. Such devices vary widely in complexity and ease of use/maintenance. Phallic devices have been employed for years by women who do not have a sex partner or who prefer to engage in safe sex practices. Vaginal substitutes have likewise been used by males lacking a female partner.

The AIDS epidemic, along with increasing problems with traditional sexually transmitted diseases, has resulted in an increase in popularity of adult sex and novelty toys. Additionally, a more open attitude towards discussing sex has resulted in a greater willingness for couples to include sex toys to add variety and interest to their sex life.

Devices directed to aiding sexual stimulus have traditionally been hand operated or electrically activated. Some are complex in structure, difficult to assemble from storage, and tedious for teardown and post-use cleaning. The hand-held devices can be cumbersome to use, and frequently result in user exhaustion of the hands and arms/legs. Those devices that are clamped in position can be too restrictive—as well as dangerous if not properly used. A need exists for a simple device to improve sexual relations between consenting adults that can effectively multiply the manual energy added by the users, without resort to requirements for electrical power.

### DISCLOSURE OF THE INVENTION

An adult sexual aid, comprising: a first pipe and a second pipe, said second pipe partially received within said first pipe and in sliding relation thereto; an elastic member received within said first pipe and interengaging with said second pipe in a manner biasing said second pipe away from said first pipe; a support arm attached to said first pipe, said support arm having a sex toy mounting surface formed thereon at a location spaced from said first pipe, whereby a user may selectively mount an adult toy upon said mounting surface prior to making use of said adult sexual aid.

An adult sexual apparatus consists of a nested pipe and spring mechanism with an attached bracket that is used to mount adult sex toys. An adjustable grip is located on the upper pipe, with a rubber stopper is located at the bottom of the lower pipe to provide stability.

A compression spring is located within the two pipes, with an inner spring stop in the lower pipe enabling a user to generate a reciprocating motion in the upper pipe. Attaching the bracket holding the sex toy to the upper pipe causes the bracket and sex toy to likewise reciprocate, which movement simulates the motions associated with actual sex.

A user initially grabs the upper ring grip and pushes/pulls it down, forcing the upper pipe to move down, compressing the spring. As the user lessens the downward pressure, the compressed spring pushes up against the upper pipe, forcing it to upwardly rebound towards its original, extended position. The compressible spring enables the device to be operated in a plunging, bouncing, thrusting, and penetrating motion—one that can be manually operated at a desired speed.

Attachment of the sex toy, such as a synthetic penis, to an adjustable bracket that is itself attached to the upper pipe, enables the user's hands and arm movements to simulate a thrusting-type motion that is transferred to the sex toy, reciprocating at a speed and in a manner corresponding to the movements and speed of a user's hands and arms—thereby enhancing the user's enjoyment.

These and various other advantages and features of the present invention are pointed out with particularity in the claims. Reference should also be had to the drawings that form a further part hereof, as well as to the accompanying descriptive matter in which are illustrated and described various examples in accordance with the invention.

### BRIEF DESCRIPTION OF THE DRAWINGS

FIG. 1 is a perspective view showing a female user of an adult sexual aid device in accordance with the present invention.

FIG. 2 is a partially exploded view, in perspective, showing an embodiment of the adult sexual aid device of FIG. 1.

FIG. 3 is an exploded view, in perspective, of an adult sexual aid device.

FIG. 4 is a partial elevated cross-sectional view of a sex toy retaining cup shown retaining an adult sex toy.

FIG. 5 is a partial elevated cross-sectional view of a sex toy retaining cup shown retaining an adult sex toy of alternative diameter to that shown in FIG. 4.

### DETAILED DESCRIPTION OF THE PREFERRED EMBODIMENTS

Reference is now made to the drawings wherein like numerals refer to like parts throughout. In FIG. 1, a female user 10 is shown holding an adult sexual aid device 14. Consisting of an upper pipe 22 and a lower pipe 26, that together form a compressible rod, the adult sexual aid device 14 is shown held by the user 10 using an upper grip 28 and a lower-mounted, gripping ring passion grip 32. A rubber stopper 36 is located at the bottom of the lower pipe 26 and is designed to provide stability to the sexual aid device 14 by gripping the surface upon which it rests.

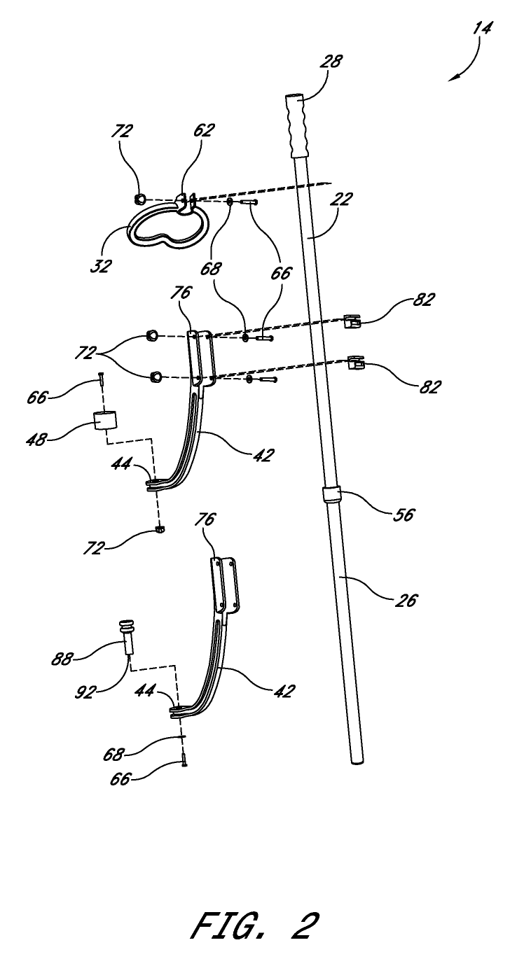

A cradle support arm 42 is attached to the upper pipe 22 and extends downwardly in a curved manner, terminating at a cradle mounting surface 44 (shown better in FIG. 2). As shown in FIG. 1, a cup mount 48 is shown received upon the cradle mounting surface 44, with the cup mount retaining an adult toy 52 in appropriate position for use with the adult sexual aid device 14.

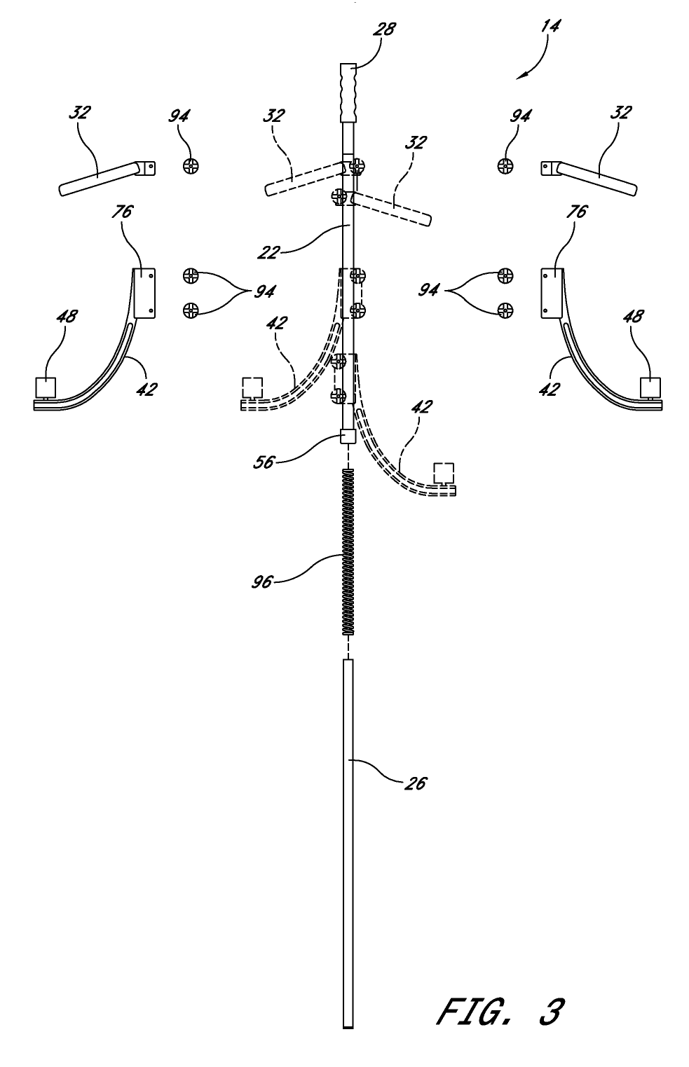

Returning to FIG. 2, the upper pipe 22 and the lower pipe 26 are selectively joined together using a pipe retaining ring 56. The connection of the upper pipe 22 and the lower pipe 26 is maintained by the retaining ring 56 in a manner that permits the lower pipe 26 to be slidably received within the upper pipe 22. The manner of its operation will be hereinafter discussed in the context of FIG. 3.

In FIG. 2, the upper grip 28 is shown as received by an upper end of the upper pipe 22 in a manner similar to the reception of handgrips on a bicycle handle. The passion grip 32 is attached to the upper pipe 22 using a circular retaining bracket 62 formed in the passion grip 32. A threaded pin 66, a washer 68, and a nut 72 are used to secure the circular retaining bracket 62 to the upper pipe 22 at a desired position.

A double-flanged bracket 76 is formed at an upper end of the cradle support arm 42 and is used to fasten the latter to the upper pipe 22. It is important that the cradle support arm be securely so fastened, and thus a pair of threaded pins 66, washers 68, and nuts 72 are used to form the connection. A pair of spacers 82 are received between the opposing flanges in a manner to prevent the over-tightening thereof.

The cradle mounting surface 44 is located on the cradle support arm 42 at an end opposite that of the double-flanged bracket 76, and is designed to provide support for the adult toy 52 (not shown in FIG. 2). Two alternative mechanisms for attachment of the adult toy 52 are provided, the cup mount 48 and a pin mount 88. The threaded pin 66 and the nut 72 are used to attach the cup mount 48 to the cradle mounting surface 44. The pin mount 88 is attached to the cradle mounting surface 44 using the threaded pin 66 and the washer 68 that are received within a threaded opening formed in a base surface 92 of the pin mount 88.

The adult sexual aid 14 can be employed in a number of ways, and can be modified for utilization by more than one person. For example, in FIG. 3, a pair of passion grips 32 are attached to the upper pipe 22 opposite one another, as are a pair of cradle support arms 42. A plurality of threaded fasteners 94 are used to secure the various attachments to the upper pipe 22. It is to be understood and appreciated that the adult sexual aid 14 can be further modified to enable use by additional persons, beyond that illustrated in FIG. 3.

Also shown in FIG. 3 is a spring 96 that is received within the upper pipe 22 when the adult sexual aid 14 is fully assembled. The spring 96 is coated with an odorless grease to enhance its performance and minimize any noise produced as the upper pipe 22 reciprocates during use.

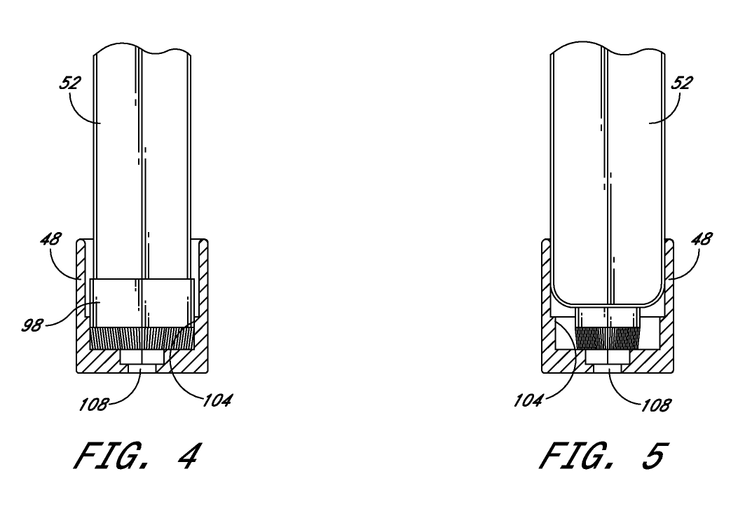

The adult sexual aid 14 is intended for use with a number of different types of phallic devices, including some that are battery powered. In FIG. 4 a battery power pack 98 is shown attached to a lower end of the adult toy 52. The cup mount 48 has an inner seat 104 that is designed to receive the battery power pack 98 and aggressively retain it during use of the adult sexual aid 14 (not shown in FIG. 4). A cup fastener 108 is used to securely attach the cup mount 48 to the cradle mounting surface 44 of the cradle support arm 42 (neither shown in FIG. 5).

In FIG. 5 the adult toy 52 is shown having a larger diameter, and without any powered pack. The cup mount 48 includes outer, extended walls 112 of a diameter intended to aggressively grip the adult toy 52, securing it in place during use of the adult sexual aid 14. Again the cup fastener is used to attach the cup mount 48 to the cradle support arm 42 (not shown in FIG. 5).

By way of example, and not limitation, where the overall length of the adult sexual aid device is 51 inches long, with the upper pipe 22 having length 29½ inches and the lower pipe having length 33½ inches. They are both preferably fabricated out of aluminum, of thickness 1/16 of an inch. The upper pipe has an outer diameter of 1 inch and the lower pipe and outer diameter of ⅞ of an inch. The pipe retaining ring 56 is preferably fabricated out of rubber and is ⅞ of an inch in length with an outer diameter of 1¼ inches. The spring 96 is preferably fabricated out of piano steel, with a length of 16 inches and an outer diameter of 0.91 inches. A food grade grease is appropriate for lubricating the spring. Upon maximum compression of the spring, the overall length of the adult sexual aid device reduces from its normal 51 inches to a compressed length of 39 inches.

The upper grip 28 is preferably nonporous, and is 4 to 7 inches in length. This grip is slidably received by the upper end of the upper pipe 22. An adhesive can be used to fixedly secure the upper grip 28 to the upper pipe 22. The passion grip 32 is preferably fabricated out of ST (super tough) 801 Nylon and is 6½ inches in diameter—with the overall width and length of 6½ and 4½ inches.

The cradle support arm 42 is also preferably fabricated out of ST 801 Nylon, and measures 18 inches in length and ⅝ inches in width (having enlarged bracket/support surfaces on each end), having a compound radius of curvature of 85 degrees. The threaded fasteners 94 used to attach the passion grip 32 and the cradle support arm 42 to the upper pipe 22 are preferably fabricated out of Nylon or steel.

The cup mount 48 is preferably fabricated out of ST 801 Nylon and has an outer diameter of 1¾ inches and length of 1⅝ inches. The inner diameter of the cup mount is 1½ inches, which reduces to an inner diameter of 1⅜ inches within the inner seat 104.

My invention has been disclosed in terms of a preferred embodiment thereof, which provides an adult sexual aid that is of great novelty and utility. Various changes, modifications, and alterations in the teachings of the present invention may be contemplated by those skilled in the art without departing from the intended spirit and scope thereof. It is intended that the present invention encompass such changes and modifications.

I claim:

1. An adult sexual aid, comprising:
a first pipe and a second pipe, said second pipe partially received within said first pipe and in sliding relation thereto;
an elastic member received within said first pipe and interengaging with said second pipe in a manner biasing said second pipe away from said first pipe;
a support arm attached to said first pipe, said support arm having a sex toy mounting surface formed thereon at a location spaced from said first pipe,
whereby a user may selectively mount an adult toy upon said mounting surface prior to making use of said adult sexual aid.

2. An adult sexual aid according to claim 1, and further comprising a hand grip received by said first pipe.

3. An adult sexual aid according to claim 2, wherein said hand grip comprises a linear grip received by a first end of said first pipe.

4. An adult sexual aid according to claim 2, wherein said hand grip comprises a gripping ring attached to said first pipe at a distance spaced from said first end.

5. An adult sexual aid according to claim 4, wherein a plurality of hand grips are attached to said first pipe.

6. An adult sexual aid according to claim 2, wherein said support arm extends from a location of attachment to said first pipe in a curvilinear manner.

7. An adult sexual aid according to claim 6, and further comprising a cup mount attached to and received by said mounting surface of said support arm.

8. An adult sexual aid according to claim 6, and further comprising a pin mount attached to and received by said mounting surface of said support arm.

9. An adult sexual aid according to claim 2, wherein said elastic member comprises a spring.

10. An adult sexual aid according to claim 9, wherein said first pipe is an upper pipe and said second pipe is a lower pipe, and wherein upon application of a downward force applied to said upper pipe, a portion of said lower pipe is compressively received within a portion of said upper pipe.

11. An adult sexual aid, comprising:
a compressible rod;
a handle attached to said compressible rod;
a cradle support arm attached to said compressible rod, said cradle support arm having a mounting surface formed thereon at a location spaced from said compressible rod;
an adult toy received by and attached to said mounting surface.

12. An adult sexual aid according to claim 11, and further comprising a second handle attached to said compressible rod at a location spaced from an end thereof.

13. An adult sexual aid according to claim 12, wherein said second handle comprises a gripping ring.

14. An adult sexual aid according to claim 13, wherein a plurality of said second handles are attached to said compressible rod.

15. An adult sexual aid according to claim 14, wherein said cradle support arm extends from said compressible rod in a curvilinear manner.

16. An adult sexual aid according to claim 15, wherein said compressible rod comprises a nested pair of pipes and an internally mounted elastic member received therein, said elastic member outwardly biasing said nested pair of pipes.

17. An adult sexual aid according to claim 16, wherein said elastic member is a spring.
